(19) United States
(12) Patent Application Publication
(10) Pub. No.: US 2025/0059503 A1
(43) Pub. Date: Feb. 20, 2025

(54) DEVICE AND METHOD FOR CREATING ORGANOIDS, AND CELL CULTURE

(71) Applicant: TECHNISCHE UNIVERSITÄT ILMENAU, Ilmenau (DE)

(72) Inventors: Andreas SCHOBER, Erfurt (DE); Jörg HAMPL, Erfurt (DE); Dana BRAUER, Elgersburg (DE); Patrick MAI, Fraubrunnen (CH); Leon KAYSAN, Bischofsheim (DE); Lan Vi NGUEN, Berlin (DE); Insa SCHRÖDER, Darmstadt (DE)

(21) Appl. No.: 18/722,012

(22) PCT Filed: Jun. 20, 2022
(86) PCT No.: PCT/EP2022/066681

§ 371 Date: Jun. 20, 2024

(51) Int. Cl. C12N 5/00; C12M 1/12; C12M 3/06

(52) U.S. Cl. CPC C12N 5/0062; C12M 23/16; C12M 25/04; C12M 25/10; C12M 25/18; C12N 2533/40; C12N 2533/90; C12N 2535/00

(57) ABSTRACT

The present invention relates a method for creating organoids, each simulating a biological organ having an organ development state. A substrate is provided, which has a plurality of cavities, each cavity being designed, in its size and form, to receive the organ to be simulated having the organ development state to be achieved. The cavities each have the form of a recess. The recesses have a length and a width, the length being greater than the width. Microfilaments are provided, each having a length which is greater than the width of the recesses and less than the length of the recesses. The individual microfilaments are arranged in the cavities. Living culturable cells are also arranged in the cavities. Conditions provided in the cavities for the culture of the cells on the individual microfilaments. The invention relates to a cell culture and a device for producing an arrangement for creating organoids.

### BACKGROUND OF THE INVENTION

The present invention relates firstly to a method for creating organoids, each organoid simulating a biological organ having an organ development state to be achieved. The invention further relates to a cell culture for creating a plurality of organoids and to a device for producing an arrangement for creating organoids.

Organoids are groups of cells cultivated in the laboratory that form organ-like structures, preferably on the basis of stem cells. Certain conditions must be met, such as contact with neighboring cells, a concentration of cell differentiation factors and the presence of extracellular matrix proteins.

In the article by Sato, T. et. al: “Single Lgr5 stem cells build crypt-villus structures in vitro without a mesenchymal niche” in Nature 459, pages 262-265, 10.1038/nature07935, 2009, the creation of three-dimensional organoids embedded in Matrigel is described.

In the article by Lancaster, M. and Knoblich, J. A.: “Generation of cerebral organoids from human pluripotent stem cells” in Nature protocols, 9(10), pages 2329 to 2340, 10.1007/978-1-4939-7024-7_8, 2014, the creation of cerebral ectodermal organoids is described.

The article by Lancaster, M., Corsini, N., Wolfinger, S. et al: “Guided self-organization and cortical plate formation in human brain organoids” in Nat Biotechnol 35, pages 659-666 10.1038/nbt.3906, 2017, presents a protocol according to which the formation of a neuroectoderm and differentiation in the neural direction can be improved by the use of floating microfilaments, which serve as scaffolds.

DE 10 2017 217 738 B3 concerns the culture of biological cells and tissues with organ-like function on a microphysiological scale. A method for the microphysiological co-culture of 3D organoid tissue and at least one 2D cell layer is used for this purpose.

WO 2017/121754 A1 shows a method for creating an elongate or fiber-supported multicellular collection of multipotent cells. Multipotent cells in an elongate or longitudinal arrangement should be arranged with an aspect ratio of at least 2:1. The collection should contain cells in different stages of differentiation and polar cells. According to this method, non-porous biodegradable microfilaments are used to support or polarize pluripotent cells. These cell clusters are then transferred into matrix drops to be incubated in microtiter wells in a free-floating manner, preferably under shaking culture. The disadvantages of this solution are the low yield and the size of the preferably cerebral embryoid bodies created in this way, the formation and size distribution of which is statistically distributed.

### SUMMARY OF THE INVENTION

The object of the present invention, based on the prior art, is to be able to produce organoids which each simulate a biological organ having an organ development state to be achieved. For stem cell research, for example, it should be possible to reproducibly produce organoids of the same geometric size with the same properties.

Said object is achieved by a method according to the appended claim 1 and by a cell culture according to the appended additional independent claim 13 and by a device according to the appended additional independent claim 14.

The method according to the invention is used to create organoids, each of which simulates a biological organ having an organ development state. The biological organ to be simulated is in particular an animal or human organ, such as a liver, stomach, brain or kidney. The organoids to be created do not yet fully simulate the organ, but rather each represent an organ-like substructure that replicates an organ development state of the organ, such as a neuroectoderm. The organ development state to be achieved is characterized in particular by a status of differentiation of biological cells of the organoid as well as by a form and size of the organoid. The method is used in particular to create as many identical organoids as possible at the same time. This number is preferably at least 20 and more preferably at least 50.

In one step of the method, a substrate is provided which has a plurality of cavities so that it represents a molded body. The substrate serves as a bioreactor for the creation of organoids, making it suitable for the culture of cells. The substrate is preferably made of a biocompatible material. The substrate is preferably made of polycarbonate or polylactide-co-glycolide (PLGA). One of the organoids is produced in each of the cavities so that the cells are cultured in the individual cavities. The size and form of the cavities are designed to receive the biological organ to be simulated having the organ development state to be achieved. Thus, the size and form of the cavities are selected according to the form and size of the organoids, which each simulate the biological organ having the organ development state to be achieved. The cavities therefore determine the size and form of the organoids to be created. Accordingly, in order to carry out the method, it is necessary to decide or determine the form and size of the organ having the organ development state to be achieved, so that the organoids to be simulated will largely have the same form and size as a result of the method due to the shaping by the cavities. The number of cavities in the substrate is preferably at least 20 and more preferably at least 50.

The cavities each have the form of a recess, which is formed in the substrate. The recesses each have a length and a width. The length and width lie in particular in a horizontal plane, which forms a main extension plane of the substrate. The recesses are formed in the vertical depth. The length and width are measured in particular in an uppermost horizontal plane, where the recesses preferably have their greatest extent in a horizontal direction. The length and width are preferably oriented perpendicular to one another. The length of the individual recesses is greater than their width. The recesses therefore each have an elongate form in relation to the horizontal plane. The elongate form defines the direction of the recesses, thus specifying a polarity for the culture of cells. The length is not only negligibly greater than the width. The depth of the recesses is preferably at most as great as their width. The depth of the recesses is preferably as great as their width.

In a further step, microfilaments are provided. The microfilaments are preferably made of a synthetic material. The microfilaments are preferably made of a biocompatible material. The microfilaments are preferably made of a self-dissolving material; in particular of a suture material such as is used in surgery to close wounds. The microfilaments preferably each have a rod form or the form of a cylinder. The microfilaments preferably each have the form of a straight thread portion. The microfilaments are preferably formed by a portion of a microfiber. The microfilaments each have a length. The length of the individual microfilaments is preferably more than twice the diameter of the individual microfilaments. In any case, the length of the individual microfilaments is greater than the width of the recesses. In addition, the length of the individual microfilaments is smaller than the length of the recesses. This dimensioning of the microfilaments should later ensure alignment of the microfilaments in the recesses.

In a further step, the individual microfilaments are arranged in the cavities of the substrate. As a rule, exactly one of the microfilaments should be arranged in each of the cavities. It is preferable for a majority of the cavities to each contain therein one of the microfilaments. It is preferable for a majority of the cavities to each contain therein at most one of the microfilaments. Due to the dimensioning of the microfilaments and the cavities formed by the recesses, the microfilaments are aligned in the recesses in such a way that a central axis of the microfilaments is aligned as far as possible parallel to a straight line on which the length of the respective recess is determined. The microfilaments and the recesses therefore point in the same direction in the individual recesses. The central axes of the microfilaments preferably lie in a horizontal plane in each case.

In a further step, living culturable cells are placed in the cavities of the substrate. These cells are suitable for culture and differentiation to the organoid. The cells are in particular tissue cells, progenitor cells, neural stem cells, embryonic stem cells, embryonic cancer cells and/or pluripotent stem cells. Embryonic stem cells and/or pluripotent stem cells are particularly preferred. The cells to be introduced into the cavities comprise cells of at least one cell type, although cells of several cell types are also possible. A sufficient number of cells must be introduced into the cavities so that said cells can be cultured into the organoid to be obtained.

In a further step, conditions are provided in the cavities that are suitable for culturing the cells on the individual microfilaments in the cavities. These conditions preferably include a culture medium, a water content, a temperature, a pressure and/or electromagnetic irradiation. Due to the conditions provided, the cells in the cavities reproduce and differentiate. As a result, an organoid is formed in each cavity, the form and size of which organoids was determined by the form and size of the cavity forming the recess, wherein the microfilament located there served as a structure to support the polarization of the cells.

A particular advantage of the method according to the invention is that it allows the production of a large number of identical organoids in an organoid factory. The organoids can be reproducibly produced with the same size and the same properties. The method allows the generation of large quantities of identical organoids for use in the fields of therapy development, pharmaceutical research and stem cell research. The method enables the mass production of organoids for industrial use. The cell differentiation state required or desired for culture and the polarity or polarization orientation can be specified in order to induce organoids with the same geometric dimensions as far as possible.

The substrate to be provided preferably has the form of a plate, which is designed to be arranged in a horizontal plane. The cavities are distributed in the horizontal plane. The cavities protrude vertically into the depth. The substrate to be provided preferably has a flat upper side from which the cavities protrude into the depth. The substrate is particularly preferably formed by a film in which the cavities are molded downwards. In preferred embodiments, the substrate formed in particular by the film is porous, at least in the cavities, so that it is permeable to gases and water, in particular also to fluids such as a cell culture medium. For this purpose, the film is preferably ion-blasted and then etched. The pores each have a diameter which is preferably at most 10 μm and further preferably at most 5 μm. The diameter of the pores is preferably at least 0.5 μm and more preferably at least 1 μm. The film has a thickness which is preferably between 10 μm and 500 μm and more preferably between 50 μm and 100 μm. The substrate formed in particular by the film has dimensions in the horizontal plane that are preferably between 0.5 cm and 15 cm and further preferably between 5 cm and 10 cm. The substrate formed in particular by the film preferably has the form of a rectangle or more preferably the form of a circle in the horizontal plane.

In preferred embodiments, at least a predominant proportion of the cavities have the same design. Likewise, a predominant proportion of the microfilaments are preferably of the same design. Preferably, all of the cavities have the same design. Preferably, all of the microfilaments are of the same design. The equalities stated here include manufacturing tolerances. The method is suitable for the mass production of identical organoids.

In preferred embodiments, the length of the recesses is at least 1.1 times their width. In further preferred embodiments, the length of the recesses is at least one and a half times their width. In other preferred embodiments, the length of the recesses is at least twice as long as their width. Preferably, the length of the recesses is no more than five times their width.

The length of the recesses is preferably between 0.1 mm and 10 mm. The length of the recesses is further preferably between 0.2 mm and 1.5 mm.

The width of the recesses is preferably between 0.05 mm and 8 mm. The width of the recesses is preferably between 0.1 mm and 0.7 mm.

The depth of the recesses is preferably between 0.05 mm and 3 mm. The depth of the recesses is further preferably between 0.1 mm and 0.7 mm.

In relation to the horizontal plane, the recesses preferably have an oval form or the form of an ellipse or the form of a rectangle with rounded corners or a kidney form. The recesses each have a base, which can be substantially flat. However, the form of the recesses in the depth can also be a continuation of the form in the horizontal plane. This means that the recesses can each have the hollow form of half an ellipsoid.

The cavities formed by the recesses are preferably evenly distributed in the horizontal plane of the substrate. The cavities formed by the recesses are preferably distributed in a spiral in the horizontal plane of the substrate, which preferably has the form of a circle in the horizontal plane.

The microfilaments preferably each have a hydrophobic surface. The microfilaments are preferably made of a self-dissolving suture material. The microfilaments preferably consist of a self-dissolving suture material, which is formed by a copolymer of glycolide and lactide. The microfilaments preferably consist of a polylactide-co-glycolide (PLGA). The microfilaments preferably consist of spun fibers, with spaces preferably remaining between the fibers. In preferred embodiments, the microfilaments are porous so that gases and water can flow through them. The pores each have a diameter which is preferably at most 5 μm and further preferably at most 3 μm. The diameter of the pores is preferably at least 0.2 μm and more preferably at least 0.6 μm. The pores are preferably formed in the longitudinal direction and/or in the transverse direction of the microfilaments.

The length of the microfilaments is preferably between 0.05 mm and 10 mm. The length of the microfilaments is preferably between 0.2 mm and 1.5 mm.

The diameter of the microfilaments is preferably between 0.5 μm and 200 μm. The diameter of the microfilaments is further preferably between 2 μm and 20 μm.

Before arranging the individual microfilaments in the cavities, the microfilaments are preferably degassed, for which purpose they are preferably placed in ethanol and then washed with distilled water. The microfilaments are then dried.

The individual microfilaments are preferably arranged in the cavities by placing the microfilaments in a transport liquid and flushing them into the cavities with this liquid. The transport liquid is preferably distilled water. The transport liquid containing the microfilaments is preferably flushed into the cavities using a pump. The pump is preferably formed by a peristaltic pump. Preferably, the transport liquid containing the microfilaments is conveyed in a circuit comprising the substrate and the pump. Preferably, the transport liquid is pumped through the pores in the cavities in the substrate so that the individual microfilaments enter the cavities and remain there due to their size, while the transport liquid passes through the pores of the substrate. The transport liquid containing the microfilaments is flushed onto the substrate from above. The transport liquid containing the microfilaments is distributed over the cavities so that the microfilaments are also distributed evenly over the cavities, which means that at least one of the microfilaments reaches almost all of the cavities. The transport liquid containing the microfilaments is conveyed at a low flow rate, preferably between 1 μl/min and 100 μl/min. The transport liquid containing the microfilaments is conveyed for a period of time which is preferably between 10 min and 100 min. The remaining transport liquid is then preferably pumped out and the substrate with the microfilaments is dried.

Preferably, at least half of the cavities each contain therein one of the microfilaments. Preferably, at least half of the cavities contain at most one of the microfilaments.

Preferably, at least 90% of the cavities contain therein one of the microfilaments. Preferably, at least 90% of the cavities contain therein at most one of the microfilaments.

The arrangement of the living culturable cells in the cavities of the substrate is preferably carried out after the individual microfilaments have been arranged in the cavities of the substrate.

Before placing the living culturable cells in the cavities of the substrate, the cells are preferably placed in a medium. The medium is preferably a culture medium. The medium containing the cells is preferably filled into the cavities drop by drop, wherein the drops have a volume which is preferably less than 100 μl.

The cells to be arranged in the cavities can be present individually or in the form of agglomerates. The number of cells to be arranged in the individual cavities is preferably at least 1,000 and further preferably at least 10,000.

After the cells have been arranged in the cavities, the cells multiply and differentiate as the necessary conditions for this are provided. An organoid forms in each of the individual cavities. The formation of the organoids is significantly influenced by the microfilament arranged in the respective cavity, since the proliferating cells settle on the microfilament, so that the microfilament specifies an orientation and/or a polarity or a polarization orientation. The identically designed microfilaments in combination with the identically designed cavities also ensure that the organoids are formed in the same way and are as similar as possible as a result. The conditions for culturing the cells in the wells are maintained for a period of time until the organoids have reached the organ development state to be achieved. This period is preferably at least 1 day and preferably no more than 30 days.

In preferred embodiments, the substrate with the microfilaments and cells therein is arranged in a bioreactor to provide the conditions for culturing the cells in order to supply the cells with fluid. A multi-channel fluidic supply to the cells in the cavities is preferred. Further shaping and/or supporting structures are preferably arranged in the cavities in order to control the formation of the organoids. Mechanical, tactile, magnetic and/or electrical stimuli are also preferably applied to the cells in the cavities in order to control the formation of the organoids.

In preferred embodiments, the organoids are removed from the cavities of the substrate, preferably by rinsing, after they have reached the organ development state to be achieved. In preferred embodiments, each of the individual organoids is arranged in a shaping formed of a basal membrane-like matrix. One form of the basement membrane-like matrix is known under the brand name Matrigel. The shapings are preferably each formed by a drop. Further culturing of the organoids takes place in the basal membrane-like matrices. Alternatively or additionally, the extracted organoids are preferably arranged in a single-channel or multi-channel bioreactor arrangement, in an insert system or in other cavities, such as in a microtiter plate.

Steps of the method, in particular the arrangement of the individual microfilaments and/or the living culturable cells in the cavities, are preferably automated using a robot. The robot is also preferably used to provide the conditions for culturing the cells.

In other embodiments, no microfilaments are used. This means that no microfilaments are provided and no microfilaments are arranged in the cavities. Instead, the cells introduced in one of the cavities are glued together using a substance that glues the cells together. The substance that glues the cells together is suitable for mechanically connecting the cells to each other by adhesion forces, which fixes their positions in relation to one another and to the substrate. The aim of gluing is the same as with the microfilaments described above, namely to orient and support the polarization of the cells. The substance gluing the cells together is preferably formed by a hydrogel, which is preferably a cross-linked polymer hydrogel. The substance that glues the cells together is preferably formed by a two-component hydrogel. A first precursor represents a first component of the two-component hydrogel, while a second precursor represents a second component of the two-component hydrogel. The first precursor and the second precursor are preferably each present in the form of a solution. The first precursor and the second precursor are placed on the cells one after the other or simultaneously in order to glue the cells together. The substance gluing the cells together or its precursors is/are preferably applied to the cells using a locally dispensing system, such as a dispenser or pipetting system that can be moved in the x-y directions or preferably a dispenser or pipetting system that can be moved in the x-y-z directions. During this process, the cells are located in the cavities, and preferably a medium, in particular a culture medium, is also arranged in the cavities so that the cells are located in the medium in the cavities while they are being glued together. Otherwise, the method is preferably carried out as described above.

In other embodiments, no microfilaments are used either. This means that no microfilaments are provided and no microfilaments are arranged in the cavities. Instead, the cells are glued together using a substance that glues the cells together before they are placed in the cavities. This means that an agglomerate of the cells is created outside the cavities in advance, so that the cells are then arranged as agglomerates in the cavities. The cells are preferably glued together in a vessel. The substance that glues the cells together is suitable for mechanically connecting the cells to each other by adhesion forces, which fixes their positions in relation to one another. The aim of gluing is the same as with the microfilaments described above, namely to orient and support the polarization of the cells. The substance gluing the cells together is preferably formed by a hydrogel, which is preferably a cross-linked polymer hydrogel. The substance that glues the cells together is preferably formed by a two-component hydrogel. A first precursor represents a first component of the two-component hydrogel, while a second precursor represents a second component of the two-component hydrogel. The first precursor and the second precursor are preferably each present in the form of a solution. The first precursor and the second precursor are placed on the cells one after the other or simultaneously in order to glue the cells together. The substance gluing the cells together or its precursors is/are preferably applied to the cells using a locally dispensing system, such as a dispenser or pipetting system that can be moved in the x-y directions or preferably a dispenser or pipetting system that can be moved in the x-y-z directions. Otherwise, the method is preferably carried out as described above.

The cell culture according to the invention is used to produce a large number of organoids, each of which simulates a biological organ having an organ development state. The cell culture comprises a substrate which has a large number of cavities. The size and form of the cavities are designed to receive the biological organ to be simulated having the organ development state to be achieved. The cavities each have the form of a recess. The recesses each have a length and a width. The length is greater than the width. A microfilament is arranged in each of the individual cavities and has a length that is greater than the width of the recesses and less than the length of the recesses. Culturable cells are arranged on the microfilaments in the cavities of the substrate and multiply and/or differentiate there in order to form the organoids having the organ development state to be achieved. The cell culture was preferably created using the method according to the invention or one of the preferred embodiments of the method according to the invention described. The cell culture preferably also has features which are indicated in conjunction with the method according to the invention or one of the preferred embodiments of the method according to the invention described.

The device according to the invention is used to produce an arrangement for creating organoids. The organoids each simulate a biological organ having an organ development state. The device initially comprises at least one substrate on which the organoids are to be created later. The substrate has a large number of cavities, the size and form of which are designed to receive the biological organ to be simulated having the organ development state to be achieved. The cavities each have the form of a recess. The recesses each have a length and a width, the length being greater than the width. The device further comprises a reservoir for storing a transport liquid containing a plurality of microfilaments. The microfilaments each have a length which is greater than the width of the recesses and less than the length of the recesses. The reservoir is preferably formed by a hose portion or a vessel. The device additionally comprises a flushing device which is used to flush the transport liquid containing the microfilaments into the cavities of the substrate so that one of the microfilaments remains in each of the cavities, if possible. For this purpose, the flushing device comprises a substrate holder for holding the substrate. The substrate can be clamped tightly in the substrate holder. The flushing device comprises an inlet for the transport liquid containing the microfilaments, so that the transport liquid containing the microfilaments can be fed into the flushing device. The inlet flows into a volume above the substrate holder. This volume is formed within the flushing device above the substrate holder so that it is located above the cavities of the substrate held in the substrate holder. This allows the transport liquid containing the microfilaments to flow through the inlet into the cavities. The device also comprises a pump for conveying the transport liquid containing the microfilaments from the reservoir into the inlet of the flushing device. This means that the transport liquid containing the microfilaments can be conveyed from the reservoir through the inlet of the flushing device into the cavities of the substrate in the substrate holder.

In preferred embodiments of the device, a circuit is formed for the transport liquid containing the microfilaments. For this purpose, the flushing device has a volume under the substrate holder, which opens out into an outlet of the flushing device. The outlet is preferably connected to the reservoir or the pump via a hose. The reservoir or the pump is preferably connected to the inlet of the flushing device via a hose. The reservoir and the flushing device and, if necessary, the pump form the circuit for the transport liquid containing the microfilaments. The transport liquid containing the microfilaments flows from the volume located in the flushing device above the substrate holder through the pores in the substrate into the volume located in the flushing device below the substrate holder.

The flushing device is preferably designed as a block with an upper block part and a lower block part, with the substrate holder being formed between the upper block part and the lower block part. The block parts are fixedly connected to one another via a detachable connection. To insert the substrate into the substrate holder and to remove the substrate from the substrate holder, the connection between the block parts must be loosened. The inlet is preferably located at the top of the block. The outlet is preferably located at the bottom of the block. The upper block part and/or the lower block part preferably have a vent. However, the flushing device can also have a fundamentally different design as long as the inlet opens directly or indirectly into the volume above the substrate holder so that the transport liquid containing the microfilaments reaches the cavities of the substrate located in the substrate holder.

The pump is preferably formed by a peristaltic pump. However, other types of pumps can also be used.

The device preferably also comprises the transport liquid containing the microfilaments. The device preferably also has features which are indicated in conjunction with the method according to the invention or one of the preferred embodiments of the method according to the invention described.

### BRIEF DESCRIPTION OF THE DRAWINGS

Further details and developments of the invention are apparent from the following description of preferred embodiments of the invention, with reference to the drawing. Shown are:

FIG. 1: shows a substrate to be provided for a preferred embodiment of a method according to the invention;

FIG. 2: shows a microfilament to be provided for a first preferred embodiment of the method according to the invention;

FIG. 3: shows a microfilament to be provided for a second preferred embodiment of the method according to the invention;

FIG. 4: shows a microfilament to be provided for a third preferred embodiment of the method according to the invention;

FIG. 5: shows a preferred embodiment of a device according to the invention;

FIG. 6: shows microfilaments introduced into a substrate during the method according to the invention;

FIG. 7: shows a preferred embodiment of a cell culture according to the invention; and

FIG. 8: shows organoids created by the method according to the invention.

### DETAILED DESCRIPTION OF THE INVENTION

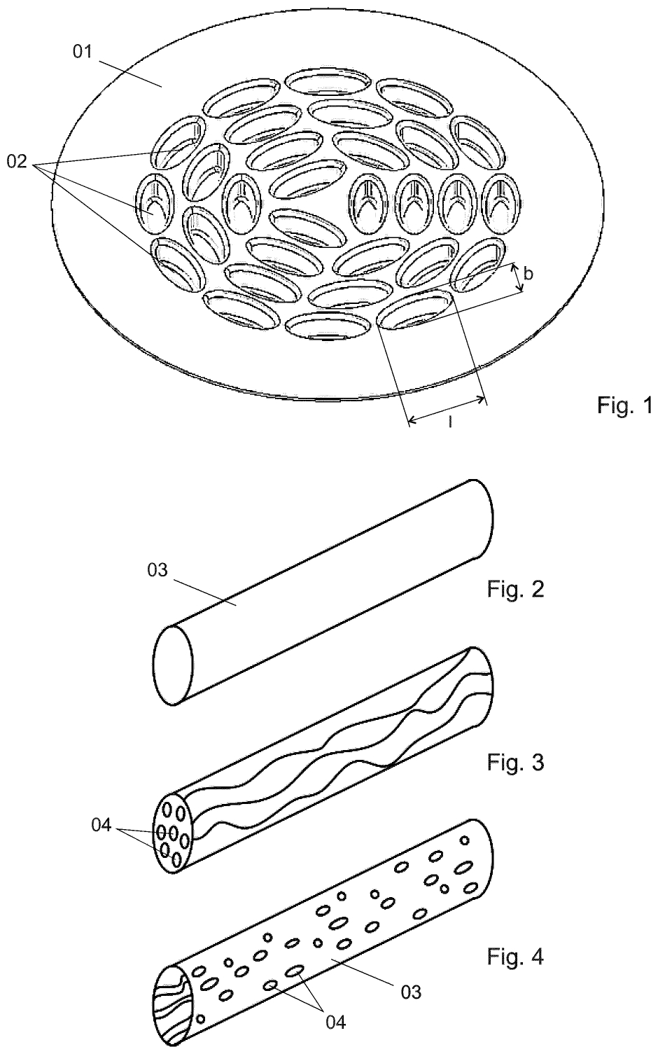

FIG. 1 shows a substrate 01 to be provided for a preferred embodiment of a method according to the invention. The substrate 01 is formed by a molded film made of polycarbonate and has a large number of identical cavities 02 in the form of recesses. The cavities 02 have an elliptical cross-section in the main extension plane of the substrate 01. The cavities 02 formed by the recesses each have a length l and a width b in the main extension plane of the substrate 01. In the example shown, the length l is approximately twice as large as the width b. The length l can be 1 mm, for example, while the width can be 0.5 mm. The substrate 01 has pores (not shown) at least in the cavities 02, so that a liquid can pass from an upper side of the substrate 01 to a lower side of the substrate 01, and vice versa. The pores each have a diameter of 4 μm, for example.

FIG. 2 shows a microfilament 03 to be provided for a first preferred embodiment of the method according to the invention. A plurality of the same microfilaments 03 must be provided in order to arrange them in the cavities 02 (shown in FIG. 1) of the substrate 01 (shown in FIG. 1). The microfilaments 03 can be provided, for example, by cutting spun fibers to size. The microfilaments 03 each have a length that is greater than the width b of the cavities 02 (shown in FIG. 1) and less than the length l of the cavities 02 (shown in FIG. 1). The length of the microfilaments 03 is 0.8 mm, for example. The microfilaments 03 have a diameter of 10 μm, for example.

FIG. 3 shows a microfilament 03 to be provided for a second preferred embodiment of the method according to the invention. In this second preferred embodiment, the microfilaments 03 have a plurality of pores 04 in the longitudinal direction.

FIG. 4 shows a microfilament 03 to be provided for a third preferred embodiment of the method according to the invention. In this third preferred embodiment, the microfilaments 03 have a plurality of pores 04 in the transverse direction.

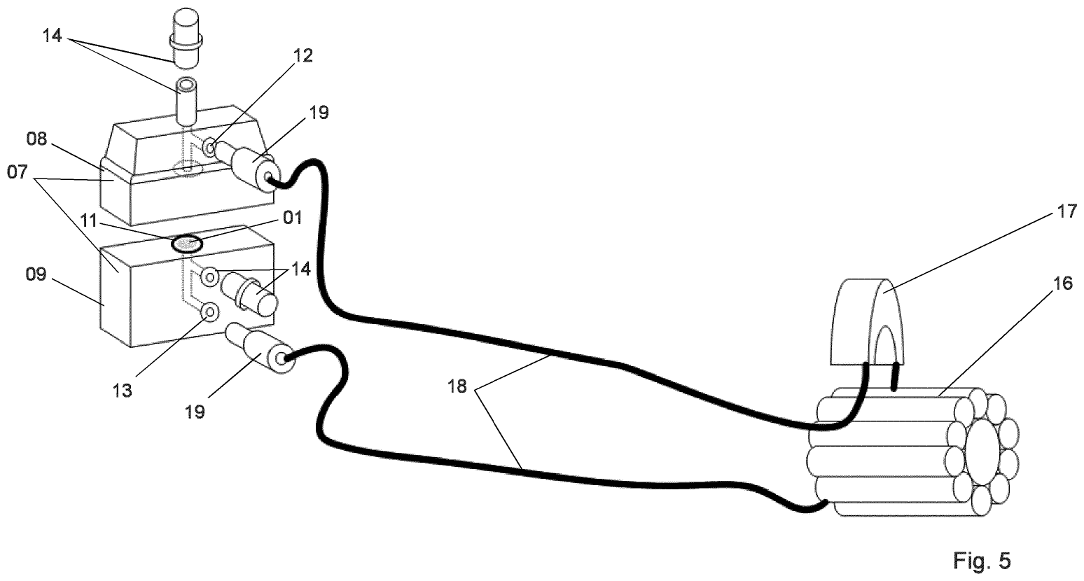

FIG. 5 shows a preferred embodiment of a device according to the invention for producing an arrangement for creating organoids 06 (shown in FIG. 8), each of which simulates a biological organ (not shown) having an organ development state. The device initially comprises a block-shaped flushing device 07, which is shown in an open state, in which an upper block part 08 of the flushing device 07 is lifted from a lower block part 09 of the flushing device 07, so that a substrate holder 11 of the flushing device 07 located between them is visible, in which the substrate 01 (shown in detail in FIG. 1) is arranged. The flushing device 07 also has an inlet 12 and an outlet 13. The substrate holder 11 with the substrate 01 is arranged fluidically between the inlet 12 and the outlet 13. The flushing device 07 also has two vents 14, which are shown in a removed state.

The device further comprises a peristaltic pump 16, which acts on a hose portion 17. The hose portion 17 is connected to hoses 18, which are connected to the inlet 12 and the outlet 13 of the flushing device 07 via hose connections 19, so that a circuit is formed.

The tubular portion 17 contains a transport liquid (not shown), which contains a plurality of the microfilaments 03 (shown in FIG. 2 to FIG. 4). The peristaltic pump 16 causes this transport liquid (not shown) containing the microfilaments 03 (shown in FIG. 2 to FIG. 4) to be conveyed through the tubes 18 and the flushing device 07, whereby it passes through the substrate 01 and the individual microfilaments 03 (shown in FIG. 2 to FIG. 4) into the cavities 02 (shown in FIG. 1), while the remaining transport liquid (not shown) flows through the pores (not shown) of the substrate 01.

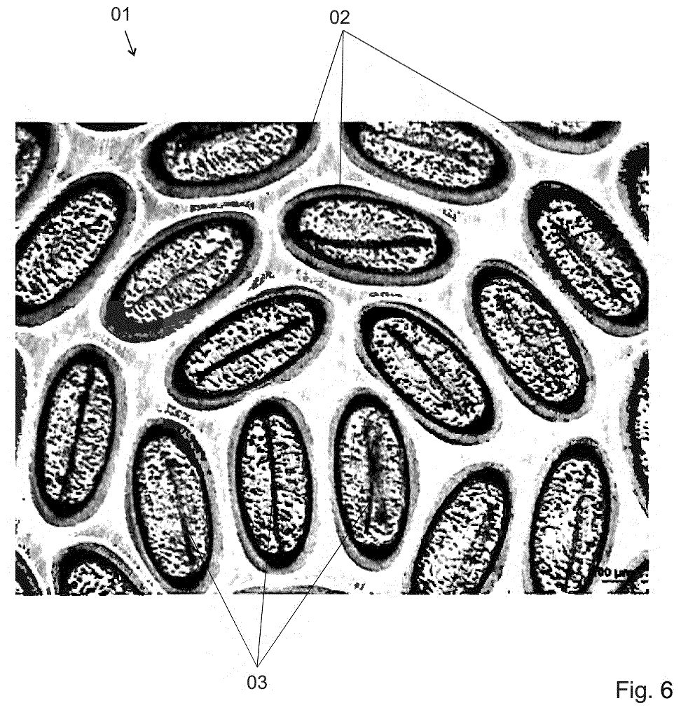

FIG. 6 shows the microfilaments 03 introduced into the cavities 02 of the substrate 01 during the method according to the invention. It can be seen that the microfilaments 03 are aligned in the longitudinal direction of the cavities 02 due to the dimensions of the cavities 02 and the microfilaments 03. The vast majority of the cavities 02 each contain exactly one of the microfilaments 03.

The microfilaments 03 were introduced into the cavities 02 of the substrate 01 by treating the substrate 01 with the device shown in FIG. 5. For this treatment, 160 of the microfilaments 03 were used, by way of example. The transport liquid (not shown) was conveyed with the peristaltic pump 16 (shown in FIG. 5) at a flow rate of, for example, 25 ml/min for a duration of, for example, 30 min. The scale bar is 100 μm long.

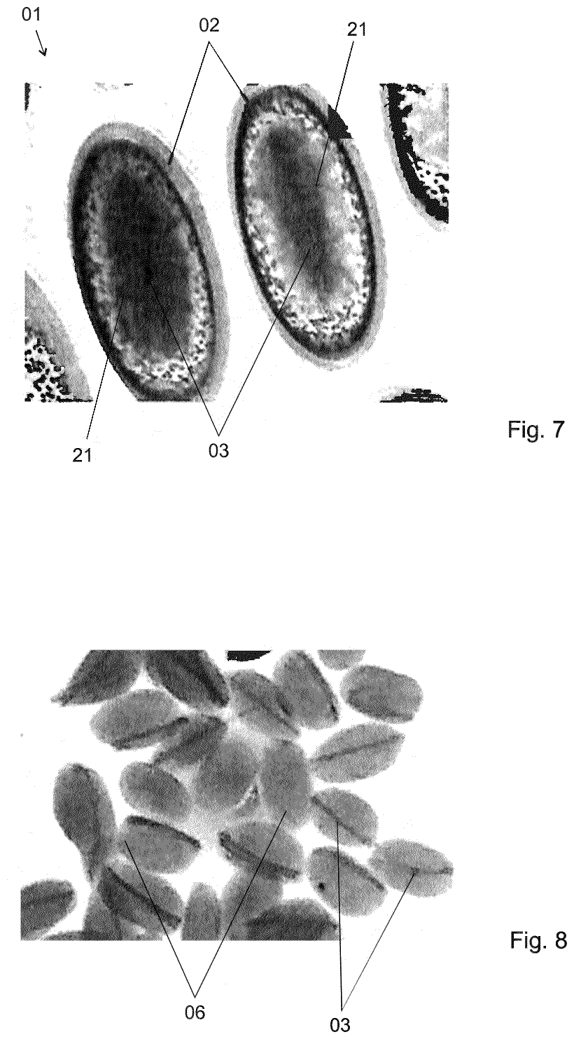

FIG. 7 shows a preferred embodiment of a cell culture according to the invention, which is produced by the method according to the invention and initially comprises the substrate 01 already described with the microfilaments 03 located in the cavities 02. In a further method step, not shown, approximately 50,000 embryonal carcinoma cells were introduced into each of the cavities 02 of the substrate 01 by way of example and conditions were provided in the cavities 02 for culturing the stem cells on the individual microfilaments 03. It can be seen that cell agglomerates 21 form on the microfilaments 03 and thus obtain a polarization. In the example shown, the cell agglomerates 21 formed after three days. The scale bar is 100 μm long. It can also be seen that the form of the cell agglomerates 21 is determined by the form of the cavities 02. After continued culture, the cell agglomerates 21 will form the organoids 06 to be created (shown in FIG. 8).

FIG. 8 shows the organoids 06 created by the method according to the invention after they have been removed from the cavities 02 (shown in FIG. 1) of the substrate 01 (shown in FIG. 1). It can be seen that the organoids 06 are largely similar due to the same initial conditions in the same cavities 02 (shown in FIG. 1) with the same microfilaments 03 (shown in FIG. 2 to FIG. 4). The organoids 06 shown by way of example are neurospheres that were cultured for 11 days. Neurospheres are collections of neural stem cells.

### LIST OF REFERENCE NUMERALS

01 Substrate
02 Cavities
03 Microfilaments
04 Pores
06 Organoids
07 Flushing device
08 Upper block part
09 Lower block part
11 Substrate holder
12 Inlet
13 Outlet
14 Vent
16 Peristaltic pump
17 Hose portion
18 Hoses
19 Hose connection
21 Cell agglomerates

1. A method for producing organoids (06) each simulating a biological organ having an organ development state, wherein the method comprises the following steps:
-providing a substrate (01) which has a plurality of cavities (02) which are each designed, in their size and form, to receive the biological organ to be simulated having the organ development state to be achieved, wherein the cavities (02) each have the form of a recess, wherein the recesses each have a length and a width, and wherein the length is greater than the width;
providing microfilaments (03) which each have a length which is greater than the width of the recesses and less than the length of the recesses;
arranging the individual microfilaments (03) in the cavities (02) of the substrate (01);
arranging living culturable cells in the cavities (02) of the substrate (01); and
providing conditions in the cavities (02) for the culture of the cells on the individual microfilaments (03) in the cavities (02).

2. The method according to claim 1, characterized in that at least a predominant proportion of the cavities (02) are of the same design, and in that at least a predominant proportion of the microfilaments (03) are of the same design.

3. The method according to claim 1, characterized in that the substrate (01) to be provided is porous at least in the cavities (02).

4. The method according to any of claims 1, characterized in that the length of the recesses is at least one and a half times as great as their width.

5. The method according to claim 4, characterized in that the length of the recesses is between 0.2 mm and 1.5 mm.

6. The method according to claim 1, characterized in that the recesses have an oval form, the form of an ellipse, the form of a rectangle with rounded corners or a kidney form in relation to a horizontal plane.

7. The method according to claim 1, characterized in that the microfilaments (03) are porous.

8. The method according to claim 7, characterized in that the microfilaments (03) consist of polylactide-co-glycolide.

9. The method according to claim 1, characterized in that the microfilaments (03) have a diameter which is between 2 μm and 20 μm.

10. The method according to claim 1, characterized in that the individual microfilaments (03) are arranged in the cavities (02) by arranging the microfilaments (03) in a transport liquid and flushing them into the cavities (02) with this transport liquid.

11. The method according to claim 1, characterized in that the cells are formed by tissue cells, progenitor cells, neural stem cells, embryonic stem cells, embryonic cancer cells and/or pluripotent stem cells.

12. The method according to claim 1, characterized in that the organoids (06), after they have reached the organ development state to be achieved, are removed from the cavities (02) of the substrate (01) and are each arranged in a shaping of a basal membrane-like matrix.

13. A cell culture for producing a plurality of organoids (06), each of which simulates a biological organ having an organ development state; wherein the cell culture comprises a substrate (01) which has a plurality of cavities (02) which are each designed, in their size and form, to receive the biological organ to be simulated having the organ development state to be achieved, wherein the cavities (02) each have the form of a recess, wherein the recesses each have a length and a width, wherein the length is greater than the width, wherein a microfilament (03) is arranged in each of the individual cavities (02), which microfilament has a length which is greater than the width of the recesses and less than the length of the recesses; and wherein culturable cells (21) are arranged on the microfilaments (03) in the cavities (02) of the substrate (01).

14. A device for producing an arrangement for creating organoids (06) each simulating a biological organ having an organ development state; wherein the device comprises the following components:
a substrate (01) which has a plurality of cavities (02) which are each designed, in their size and form, to receive the biological organ to be simulated having the organ development state to be achieved, wherein the cavities (02) each have the form of a recess, wherein the recesses each have a length and a width, wherein the length is greater than the width, and wherein the width and the length of the recess are adapted to the microfilaments (03) to be received, in such a way that the microfilaments (03) each have a length which is greater than the width and smaller than the length of the recesses;
a reservoir (17) for storing a transport liquid containing a plurality of microfilaments (03);
a flushing device (07) with a substrate holder (11) for holding the substrate (01), wherein the flushing device (07) comprises an inlet (12) for the transport liquid containing the microfilaments (03), which inlet opens out into a volume above the substrate holder (11); and
a pump (16) for conveying the transport liquid containing the microfilaments (03) from the reservoir (17) into the inlet (12) of the flushing device (07), wherein the transport liquid is pumped through pores (04) in the cavities (02) in the substrate (01), so that the individual microfilaments (03) enter the cavities (02) and remain there because of their size, while the transport liquid flows through the pores (04) of the substrate.

15. The device according to claim 14, characterized in that the flushing device (07) furthermore has a volume under the substrate holder (11) which opens out into an outlet (13) of the flushing device (07), wherein the reservoir (17) and the flushing device (07) form a circuit for the transport liquid containing the microfilaments (03).
